(19) United States
(12) Patent Application Publication
(10) Pub. No.: US 2006/0177499 A1
(43) Pub. Date: Aug. 10, 2006

(54) Method for the manufacture of a pharmaceutical composition in the form of tablets containing a fibrate and tablets obtained according to the method

(76) Inventor: Jerome Besse, Listrac Medoc (FR)

Correspondence Address: ARENT FOX PLLC, 1050 CONNECTICUT AVENUE, N.W., SUITE 400, WASHINGTON, DC, 20036, US

(21) Appl. No.: 10/546,999

(22) PCT Filed: Feb. 27, 2004
(86) PCT No.: PCT/FR04/50090

§ 371 Date: Aug. 26, 2005

(30) Foreign Application Priority Data
Feb. 28, 2003 (FR) 03/02520

(51) Int. Cl. A61K 31/192; A61K 9/20

(52) U.S. Cl. 424/464; 514/571

(57) ABSTRACT

A method for the manufacture of a pharmaceutical composition containing the active ingredient fenofibrate or one of its derivatives, optionally in combination with a second active ingredient, in the form of tablets, characterized in that it comprises a compression step of the active ingredient and excipients by means of a dry method of granulation

The present invention relates to the field of manufacture of novel galenic forms containing the active ingredient fenofibrate or one of its derivatives, optionally in the form of a combination with a second active ingredient.

Isopropyl 2-[4-(4-chlorobenzoyl)phenoxy]-2-methylpropionate, the international nonproprietary name (INN) of which is fenofibrate, is a lipid lowering active ingredient. Fenofibrate is the most used active ingredient in the world for the treatment of endogenous hypercholesterolemias and hypertriglyceridemias in the adult, in isolation or in association. Its efficacy in these therapeutic indications has been widely demonstrated. Thus, administered in the long term at therapeutically efficacious doses, fenofibrate makes it possible to lower the cholesterolemia by 20 to 25% and the triglyceridemia by 40 to 50%, and does so from the first month of treatment.

In order to obtain a satisfactory cholesterol lowering effect, it is desirable to maintain a (circulating) plasma level of fenofibric acid, which is the active metabolite of fenofibrate, in a range of plasma concentrations included between about 6 and about 10 mg/l. A long-term treatment with fenofibrate is not completely devoid of adverse effects. In particular, adverse effects have been reported such as rhabdomyolysis, asthenia, an increase of blood in the urine, an increase of creatininemia, skin rashes, urticaria, nausea, vomiting, abdominal pains, diarrhoea, an increase of transaminases, hepatic toxicity, a diminution of alkaline phosphatases, a polymyositis, muscular pains, headaches and dizziness.

This is the reason why experiments have been performed for the purpose of developing galenic formulations allowing an increased bioavailability of the active ingredient fenofibrate when the latter is absorbed by the oral route, so as to diminish the dose of fenofibrate and thus diminish, as a consequence, the risks of adverse effects.

Fenofibrate is an active ingredient very poorly soluble in water and whose absorption from the digestive tract is limited. These properties of poor solubility in water of fenofibrate have been taken into account for the design and preparation of various prior art pharmaceutical formulations containing this active ingredient.

Thus, various routes have been explored in order to increase the speed of dissolution of fenofibrate and to obtain easily administrable pharmaceutical compositions in order to improve the comfort of the patient treated.

A first technical solution consisted of micronizing the fenofibrate in combination with a surfactant, as is described in the European patent application No. EP 330 532.

The manufacture of capsules containing granules of a co-micronizate of fenofibrate with an excipient, but in the absence of surfactant, has also been described in the prior art, as for example in the European patent application No. EP 1 048.295.

The major pharmaceutical form in which fenofibrate-based pharmaceutical specialities have been marketed consisted of capsules, in particular soft gelatine capsules or hard gelatine capsules containing granules of a support constituted of pharmaceutical excipients in combination with fenofibrate.

An example of a capsule containing granules consisting of a pharmaceutical excipient support in combination with a co-micronizate of fenofibrate with a surfactant is described in the American patent No. U.S. Pat. No. 4,895,726. For the preparation of the granules contained in capsules, and in order to overcome the problems related to the poor solubility of fenofibrate in water, leading to a reduced bioavailability of this active ingredient, preparation procedures have been used in which fenofibrate is suspended in a solution of a hydrophilic polymer, then the suspension is sprayed on an inert support of hydrodispersible excipients, as is described in the French patent application published under the U.S. Pat. No. 2,758,459. Such a procedure was improved by the incorporation of a cellulose derivative used as binder and solubilization additive into the aqueous suspension designed to be sprayed over the inert excipient support, as described in the PCT application published under No. WO 01/03.693.

In other pharmaceutical formulations in the form of capsules, other technical solutions designed to improve the bioavailability of fenofibrate have been developed, like (i) the granulation of fenofibrate in the presence of a liquid medium comprising a surfactant, water and an alcohol miscible with water, as described in the French patent application published under No. FR 2.783.421, (ii) the manufacture of a preconcentrate capable of forming spontaneously an oil-in-water microemulsion in contact with an aqueous medium, said preconcentrate comprising a lipophilic phase and an emulsifying system, as described in the French patent application published under No. 2.803.203, (iii) the preparation of a combination of micronized fenofibrate and a phospholipid, as described in the American patent application published under No. US 2002/0119199, and (iv) the preparation of a suspension of fenofibrate in solution in the monoethyl ether of diethyleneglycol (EMDG), as described in the European patent application No. EP 0757 911.

Fenofibrate-based pharmaceutical formulations have also been prepared in the form of tablets. Fenofibrate-based pharmaceutical formulations in the form of tablets possess the advantage, at equal weight, of being smaller than a capsule and, in addition, allow an industrial production of such pharmaceutical formulations at higher rates than those observed for the manufacture of the formulations in the form of soft capsules. For the manufacture of such tablets, and in the light of the poor water solubility of fenofibrate, a dispersed suspension of fenofibrate is first prepared in an aqueous solution of a hydrophilic polymer, such as polyvinylpyrrolidone (PVP). In a second step, either (i) the aqueous solution of hydrophilic polymer containing fenofibrate is sprayed on an inert excipient support, as described in the European patent application No. EP 1.273.293 or also in the French patent application No. FR 2.758.461, or (ii) the fenofibrate suspension, optionally co-micronized with a surfactant, is granulated by wet granulation in an aqueous solution of polyvinylpyrrolidone, before incorporation of other possible excipients, preliminary to a compression step by a wet method in order to finally obtain a tablet, as described in the French patent application No. FR 2 819 720.

As can be seen, attempts have been made in the state of the art to progressively improve the techniques of dissolution of the active ingredient fenofibrate in order to augment its bioavailability with respect to the target sites in the organism, including the production of pharmaceutical formulations in the form of tablets, these latter offering many technical advantages both from the point of view of their industrial production and from the point of view of the comfort of the patient to be treated.

There is still a need in the art to improve the technical characteristics of a pharmaceutical composition based on an active ingredient of the fibrate type, in particular fenofibrate, and it is still desirable today (i) to improve the industrial conditions of production of such pharmaceutical formulations and (ii) to manufacture such pharmaceutical formulations making it possible to obtain a bioavailability of the fibrate in vivo at least equal to that of the known compositions in order to obtain a good therapeutic efficacy of the treatment and also to minimize the adverse effects of this active ingredient.

The above objectives have been attained according to the present invention.

Surprisingly, it has been shown according to the invention that a pharmaceutical formulation based on an active ingredient of the fibrate type, in the form of tablets, can be obtained without requiring a dispersion or dissolution of the fibrate in an aqueous solution containing a hydrophilic polymer, prior to the preparation of the solid pharmaceutical support destined to be compressed in the form of tablets.

Thus, it has been shown according to the invention that a pharmaceutical composition containing a fibrate which is prepared without a dissolution step of the fibrate in aqueous solution possesses a bioavailability in vivo of the active ingredient and pharmacokinetic properties at least equivalent to those which are observed with the pharmaceutical compositions in the form of tablets obtained by wet granulation known in the state of the art.

Hence a manufacturing procedure of a pharmaceutical composition of fibrate-based tablets has been developed according to the invention which comprises exclusively steps of granulation or compaction which are performed by a dry method.

The object of the invention relates to a method for the manufacture of a pharmaceutical composition containing the active ingredient fenofibrate or one of its derivatives in the form of tablets, characterized in that it comprises the following steps:
(a) prepare a mixture of fenofibrate or one of its derivatives, optionally in the form of a combination of fenofibrate or its derivative with a second active ingredient, and at least one solid surfactant in a ratio of (i) 91% to 99% by weight of fenofibrate or its derivative, or the combination with a second active ingredient, and (ii) 1% to 9% by weight of solid surfactant(s);
(b) micronize the mixture of fenofibrate or one of its derivatives, optionally in the form of a combination with a second active ingredient, and surfactant(s) obtained in step (a), in order to obtain a micronizate of the fenofibrate or the combination with the second active ingredient, and the surfactant(s);
(c) add at least one anti-static agent to the micronizate prepared in step (b);
(d) add to the mixture obtained in step (c) at least one diluent, at least one disintegrant and at least one lubricant in order to obtain a solid mixture corresponding to the internal phase of the tablet;
(e) compress, according to a granulation step by a dry method, the solid mixture obtained in step (d) in order to obtain the final internal phase of the tablet;
(f) mix the internal phase of the tablet prepared in step (e) with an external phase comprising at least one lubricant, then compress the composition in order to obtain the pharmaceutical composition containing fenofibrate in the form of tablets.

A derivative of fenofibrate according to the invention encompasses every type of active ingredient of the fibrate type, other than fenofibrate, including 2-[4-[2-(4-chlorobenzamido)ethyl]phenoxy]-2-methylpropionic acid(bezafibrate), 2-[4-(2,2-dichlorocyclopropyl)phenoxy]-2-methylpropionic acid(ciprofibrate), 2-(4-chlorophenoxy)-2-methylpropionate of 3-dimethylcarbamoylpropyl(clofibride), 2 bis-(4-chlorophenoxy)-2-methylpropionate of hydroxyaluminium(aluminium clofibrate) and 2,2-dimethyl-5-(2,5-xylyloxy)valeric acid (Gemfibrozil). All the active ingredients of the fibrate type possess in common a lipid lowering pharmacological property and a physico-chemical property of poor solubility in an aqueous medium.

In a pharmaceutical composition prepared in conformity with the invention, fenofibrate or one of its derivatives can be contained in the form of a combination with a second active principle such as mefformin, as described in the PCT application No. WO 99/40904, cobalamine, folic acid, betaine or N-acetylcysteine as described in the German patent No. DE 1.991.0682, vitamin E, as described in the French patent application No. FR 19 95 000 126, a HMG CoA inhibitor (statin), as described in the PCT application No. WO 01 37 831 or also in the PCT application No. WO 02 34 359.

The above method, which does not comprise a dissolution step for fenofibrate or its derivative in an aqueous solution of a hydrophilic polymer, nor, consequently, a wet granulation step prior to the manufacture of the final tablet, is thus considerably simplified in its implementation compared with the previously known methods. The implementation on an industrial scale of the method according to the invention thus allows (i) saving costs, owing to the absence of a dissolution step for the fenofibrate or its derivative and (ii) a higher yield, owing to the simplicity of its implementation making it possible to increase the rate of industrial production of the tablets, compared with the methods known in the prior art. Furthermore, the method according to the invention owing to the fact that it does not include a dissolution step for fenofibrate or its derivative in an aqueous suspension, for example in an aqueous suspension containing a hydrophilic polymer, and that consequently the whole steps of said method are carried out exclusively through a dry procedure, can be carried out continuously, which increases the yield and reduces the cost of the method.

Furthermore, as is illustrated in the examples, the fibrate-based tablets obtained according to the method of the invention possess pharmacological properties at least identical, at an equal dose of fibrate, with the fibrate tablets known previously.

Preferably, in step (a) of the method, fenofibrate or its derivative and the surfactant are introduced into a mixing device, for example into a mixing device of the Turbula® type or equivalent, then the active ingredient and the surfactant(s)are mixed. Advantageously, the mixture of the two types of compounds is carried out for ten minutes, for example at a speed of 50 revolutions/min.

In a particular embodiment of step (a), fenofibrate or its derivative are in the form of a combination with a second active ingredient and the mixture of fenofibrate or its derivative with the second active ingredient and the surfactant are introduced into the mixing device.

Step (b) of the method can be carried out in conformity with the teachings of the European patent application No. EP 0330 532.

Preferably, step (b) of the method is carried out by a micronization of the fenofibrate or its derivative, optionally in the form of a combination with a second active ingredient, and of surfactant(s) by the conventional method with a jet of air, for example by using an air jet micronization apparatus of the ALPINE or JET MILL type, according to the recommendations of the manufacturer.

The preferred parameters for a micronisation on a GALETTE ALPINE 200 AS micronizing apparatus are as follows:
1 injector: 7 to 8 bars
2 crown: 4 to 6 bars
3 air flow: 180 m³/H; and
4 rate: 25 kg/h

The above conditions for step (b) of micronization of the fibrate, optionally in the form of a combination with a second active ingredient, and of surfactant(s) leads to a co-micronisate being obtained comprising the particles possessing a size comprised between 0.1 μm and 20 μm.

In general, about 90% of the particles of the micronizate have a size comprised between about 0.8 μm and about 7 μm. According to the tests, the mean size of the particles of the micronizate is comprised between 2.5 μm and 7 μm.

The mean size of the beads or particles of the micronizate can be measured by any known conventional technique. In particular, the one skilled in the art may have recourse to a laser measurement of the granulometry with a device of the Beckman Coulter or Malvern type, as described in the examples.

Advantageously, step (c) of the method is carried out by introducing the micronizate obtained in step (b) and the antistatic agent(s) into a container, mixing them, then sieving this mixture, for example with the aid of a sieve having a mesh size of 500 μm. Then the sieved mixture thus obtained is introduced into the interior of a mixer, for example of the Bohleo type.

Then, in step (d) of the method, at least one diluent and at least one disintegrant is added to the mixture obtained in step (c), then the contents are mixed with the aid of the said mixing device, for example for about twenty minutes.

Subsequently, the appropriate quantity of at least one lubricant, optionally sieved, for example with a sieve having a mesh size of about 500 μm, then the mixture is mixed again in the same mixing device, for example for about three minutes.

In step (e) of the method, compression of the solid mixture obtained in step (d) is carried out with the aid of any conventional compression device, like for example the Alexanderwerck® compression device, which may be equipped with a stainless steel grid with a mesh opening of 1.25 mm.

The compression step (e) confers advantageous pharmacotechnical characteristics on the mixture before compression, in particular as regards the flow properties, without modifying the in vitro and in vivo dissolution profiles of the finished tablet product, compared with the dissolution profiles of a comparative tablet obtained by a method that does not comprise the compression step (e). The compression step has the effect of increasing the density of the solid mixture obtained in step (d), which can consequently be easily used by gravity at the time of compression in step (f) of the method.

Steps (a) to (e) of the method enable the internal phase of the tablet to be manufactured.

In step (f) of the method, the final internal phase of the tablet prepared in step (e) is mixed with an external phase comprising at least one lubricant. Preferably, step (f) is carried out by adding the appropriate quantity of lubricant(s), optionally sieved beforehand, and the internal phase prepared in step (e) is mixed with the lubricant(s) in a conventional mixing device, for example a mixer of the Bolhe® type, for example for about three minutes.

In step (f), the covering of the surface of the final internal phase of the tablet obtained in step (e) with one or more lubricants allows to avoid the phenomenon of abrasion at the subsequent compression step of the composition in the form of tablets, i.e. friction between the pieces of the compression device and the powder to be compressed, likely to cause a blockage of the compression step, which is otherwise observed in the absence of the coating of the final internal phase by the said lubricant.

In step (f) the compression is carried out on any conventional device. For example, the one skilled in the art may have recourse to a rotating compression machine of the Kilian® type.

For the compression, it is possible in particular to use flat, round or oblong dies including dies of the type 16R16, or also curved, round dies including dies of the type 9.5 R8 or also dies of oblong format 18×8R8.

According to a first preferred aspect of step (a) of the method of the invention, the mixture of fenofibrate or its derivative and surfactant(s) consists of a ratio of (i) 95% to 98% by weight of fenofibrate or its derivative and (ii) 2% to 5% by weight of surfactant(s).

In the specific embodiment of the method in which the fenofibrate or its derivative is combined with a second active ingredient, the mixture consists of a ratio of (i) 95% to 98% by weight of the combination between fenofibrate or its derivative and the second active ingredient and (ii) 2% to 5% by weight of surfactant(s).

In a most preferred embodiment, the mixture of fenofibrate or its derivative, optionally in the form of a combination with a second active ingredient, and surfactant(s) comprise a ratio of 96.6% by weight of fenofibrate or its derivative, or of the combination, and 3.4% by weight of said surfactant(s).

The surfactant(s) is/are preferably chosen from the following surfactants: sodium lauryl sulfate, a polyoxyethylenated ester of polysorbitan, such as the monooleate, monolaurate, monopalmitate, monostearate esters, sodium dioctylsulfosuccinate (DOSS) and lecithin.

In a most preferred embodiment, the surfactant is sodium lauryl sulfate.

According to a preferred first aspect of step (b) of the method, the micronizate consists of particles having a size comprised between 0.1 and 20 μm.

Preferably, the mean size of the beads or particles of the micronizate is comprised between 2 μm and 7 μm.

According to a first preferred aspect of step (c) of the method, the antistatic agent(s) is/are added in an amount of 0.1% to 5% by weight, and preferably from 0.2% to 2% by weight with respect to the total weight of the composition.

The antistatic agent(s) enhance the flow properties of the powder and consequently facilitate the dispersion of the different constituents, including the fenofibrate or its derivative, optionally in the form of a combination with a second active ingredient, homogeneously in the mixture in order to obtain, at the end of the method, all of the tablets that contain the desired amount of the active ingredient(s) and of the various excipients.

Preferably, the antistatic agent(s) is/are added in an amount of 0.4% to 0.7% by weight, with respect to the total weight of the composition.

In a very preferred manner, the antistatic agent(s) is/are added in an amount of 0.49% by weight or 0.50% by weight, with respect to the total weight of the composition.

Preferably, the antistatic agent(s) is/are chosen from colloidal silica, magnesium silicate, talc, calcium silicate and tribasic calcium phosphate, alone or in combination.

In a most preferred embodiment, a single antistatic agent is used which consists of anhydrous colloidal silica.

According to a first preferred aspect of step (d) of the method, the diluent(s) is/are added in an amount of 40% to 80% by weight, and preferably between 50% and 75% by weight, with respect to the total weight of the composition.

By diluent is meant according to the invention an agent used to supplement the pharmaceutical composition prepared according to the method, until a predetermined total volume of the composition is obtained, which contains the selected quantity of fenofibrate or its derivative or the combination of fenofibrate or its derivative with a second active ingredient, the volume of fibrate of the combination as such being insufficient for the production of a final pharmaceutical composition, wherein the desired volume of which comprises the appropriate quantity of this active ingredient.

Preferably, at least one of the diluents also exerts the function of binder, as for example microcrystalline cellulose.

In a most preferred embodiment, the diluent(s) is/are added in an amount of 66% to 72% by weight, and better still in an amount of 68.5% to 70.5% by weight, with respect to the total weight of the composition.

The diluents preferentially used for the implementation of the method according to the invention are selected from calcium or sodium carbonate or bicarbonate, sucrose, mannitol, xylitol, sorbitol, lactose, maltitol, glucose, cellulose powder or microcrystalline cellulose, starch and its derivatives, dibasic calcium phosphate, tribasic calcium phosphate, calcium sulfate, the dextrates, the dextrins, the dextrose excipients, fructose, kaolin, lactitol.

In a most preferred embodiment, a combination of two diluents is used, microcrystalline cellulose and lactose monohydrate, respectively.

According to a second preferred aspect of step (d) of the method, the disintegrant(s) is/are added in a proportion of 1% to 20% by weight, and preferably from 3% to 6% by weight, with respect to the total weight of the composition.

In a most preferred embodient, the disintegrant(s) is/are in an amount of 4.5% to 5.5% by weight, and better still from 4.8% to 5.1% by weight, with respect to the total weight of the composition.

Preferably, the disintegrants are selected from sodium starch glycollate, sodium croscarmellose, crosslinked polyvinylpyrrolidone, sodium carboxymethylcellulose, calcium carboxymethylcellulose and low substitution hydroxypropylcellulose.

In a most preferred embodiment, sodium croscarmellose is used as disintegrating agent.

According to a third preferred aspect of step (d) of the method, the lubricant(s) is/are added in an amount of 0.1% to 2% by weight, and preferably from 0.2% to 1% by weight, with respect to the total weight of the composition.

In a most preferred embodiment, the lubricant(s) is/are added in an amount of 0.2% to 0.8% by weight, and better still in an amount of 0.5% by weight, with respect to the total weight of the composition.

According to the method, the lubricant(s) is/are selected from magnesium stearate, calcium stearate and talc.

In a most preferred embodiment, magnesium stearate is used as lubricant.

Preferably, an amount of fenofibrate or its derivative, optionally in the form of a combination with a second active ingredient, is used in step (a) of the method, such that fenofibrate or its derivative, or the combination of fenofibrate or its derivative with the second active ingredient, is present in an amount of 20% to 50% by weight, with respect to the total weight of the final pharmaceutical composition obtained in step (f).

Preferentially, fenofibrate or its derivative, optionally in the form of a combination with a second active ingredient, is used in quantities such that fenofibrate or its derivative, or the combination with the second active ingredient, is present in a proportion of 20 to 25% by weight, and better still from 22% to 24% by weight with respect to the total weight of the final composition obtained in step (f) of the method.

According to a particular embodiment of the method according to the invention, said method comprises an additional film-coating step (g) of the tablet which was obtained in step (f), the film which then covers the whole surface of the external phase of the tablet obtained at the end of step (f) allowing to protect the composition of the tablets from the external environment, for better preservation. Furthermore, the film masks the bitterness characteristic of the active ingredient fenofibrate or its derivatives. The results presented in the Examples show that the film-coating of the tablet such as obtained at the end of step (f) of the method, does cause any detectable change in the release profile of fenofibrate.

However, this film-coating is not essential, since the tablet can be well preserved by other means, like for example a packaging limiting the exchange of water vapour with the external environment.

The film-coating may be carried out in a conventional manner according to procedures known by the one skilled in the art, for example by spraying a solution of film-forming polymer on to the tablets placed in a turbine. This film-coating also makes it possible, if necessary, to colour the tablets by the addition of a coloured pigment to the solution of film-coating polymer.

For film-coating of the tablet obtained at the end of step (a) of the matter, the one skilled in the art may use, in particular, a cellulose derivative like hydroxypropylmethylcellulose, for example hydroxypropylmethyl-cellulose sold under the name Opadry® which can be under the form of an aqueous suspension at a concentration of 15% by weight with respect to the weight of the aqueous solution used for film-coating.

A preferred film-coating according to the invention consists of (i) a film-forming agent such as hydroxypropylmethylcellulose, (ii) a plasticizer such as polyethylene glycol, (iii) a diluent such as lactose and (iv) a filler which may serve as opacifier, such as titanium dioxide.

It has been shown according to the invention that the method described above makes it easy and less expensive to obtain tablets of fenofibrate or one of its derivatives, optionally in combination with a second active ingredient, which possess an in vivo pharmacokinetic profile which is at least identical with that observed with the tablets of fenofibrate prepared by a method including a wet granulation step, as in the prior art.

Maximum plasma concentration values (Cₘₐₓ), time values until the maximum plasma concentration has been reached (Tₘₐₓ) and AUC values (for the area under the plasma concentration curve) have been calculated from pharmacokinetic profiles for tablets prepared according to the invention and for capsules prepared according to a method comprising a wet granulation step, such as in the prior art. All these values have been compared in examples 3 and 4.

It is therefore another object of the invention to provide a tablet containing fenofibrate or one of its derivatives, characterized in that it has a maximum plasma concentration similar to that of a tablet prepared according to a method comprising a wet granulation step.

It is still a further object of the present invention to provide a tablet containing fenofibrate or one of its derivatives, characterized in that it has an area under the plasma concentration curve (AUC) similar to that which can be measured for a tablet prepared according to a method comprising a wet granulation step, or for a capsule.

It will be appreciated by the one skilled in the art that “similar” as used herein corresponds to mean values that are not statistically different.

The method according to the invention is particularly suitable for preparing tablets which fenofibrate concentration ranges from 30 mg to 300 mg.

Preferably, the tablet according to the invention is dosed at 200 mg and has a pharmacokinetic profile characterized by an area under the plasma concentration curve measured in vivo (AUC) of about 220000 ng.h/ml.

Preferably, the tablet according to the invention is dosed at 200 mg and has a pharmacokinetic profile characterized by a maximum plasma concentration value (Cₘₐₓ) of about 10600 ng/ml.

Preferably, the tablet according to the invention is dosed at 200 mg and has a pharmacokinetic profile characterized by a Tₘₐₓ ranging from 2.00 to 3.75 ng/ml.

It will be appreciated by the one skilled in the art that “Tₘₐₓ” as used herein corresponds to the time until the maximum plasma concentration has been reached.

A still further object of the invention consists of a tablet containing fenofibrate or one of its derivatives, wherein said tablet comprises:

(a) an internal phase constituted of:
(i) 20% to 50% by weight of fenofibrate or its derivative, optionally in the form of a combination with a second active ingredient, in the form of a micronizate with at least one solid surfactant, the said micronizate comprising (i) from 91% to 99% by weight of fenofibrate or its derivative, optionally in the form of a combination with a second active ingredient, and (ii) from 1% to 9% by weight of the said solid surfactant(s);
(ii) from 0.2% to 2% by weight of at least one antistatic agent;
(iii) from 40% to 80% by weight of at least one diluent;
(iv) from 1% to 20% by weight of at least one disintegrant; and
(v) from 0.1% to 1% of at least one lubricant;
with respect to the total weight of the said tablet; and

(b) an external phase comprising from 0.1% to 2% of at least one lubricant with respect to the total weight of the said tablet.

According to an advantageous embodiment, said tablet is characterized in that the external phase is covered with a protective varnish, preferably a hydrodispersible. polymer-based protective varnish.

As illustrated in the Examples, various pharmaceutical formulations in the form of tablets containing the active ingredient fenofibrate or one of its derivatives, and prepared in conformity with the procedure of the invention have been produced. These specific pharmaceutical compositions also form part of the invention.

The object of the invention also relates to a tablet of fenofibrate or of one of its derivatives, characterized in that it comprises:
23.09% by weight of micronized fenofibrate or of one of its derivatives;
0.82% of micronized sodium laurylsulfate;
0.49% of anhydrous colloidal silica;
29.4% by weight of microcrystalline cellulose and 38.81% by weight of lactose monohydrate;
4.9% by weight of sodium croscarmellose;
0.5% by weight of magnesium stearate;
2% by weight of a film-coating agent;

the above percentages by weight being expressed with respect to the total weight of the tablet.

The above tablet is appropriate for a pharmaceutical formulation of fenofibrate or of one of its derivatives dosed at 160 mg per tablet.

The invention also relates to a tablet of fenofibrate or of one of its derivatives, characterized in that it comprises:
23.56% by weight of micronized fenofibrate or of one of its derivatives;
0.84 by weight of sodium laurylsulfate;
0.5% by weight of anhydrous colloidal silica;
30% by weight of microcrystalline cellulose and 39.6% by weight of lactose monohydrate;
5% by weight of sodium croscarmellose; and
0.5% by weight of magnesium stearate.

The above percentages by weight being expressed with respect to the total weight of the tablet.

The tablet such as that defined above is suitable for the production of a pharmaceutical formulation of fenofibrate or of one of its derivatives dosed at 67 mg per tablet.

The invention also relates to a tablet of fenofibrate or of one of its derivatives, characterized in that it comprises:
23.56% of micronized fenofibrate or of one of its derivatives;
0.84% by weight of micronized sodium laurylsulfate;
0.5% by weight of anhydrous colloidal silica;
30% by weight of microcrystalline cellulose and 39.6% by weight of lactose monohydrate;
5% by weight of sodium croscarmellose; and
0.5% by weight of magnesium stearate.

The above percentages by weight being expressed with respect to the total weight of the final composition.

The tablet such as defined above is suitable for the production of a pharmaceutical formulation based on fenofibrate or on one of its derivatives dosed at 200 mg per tablet.

The present invention is in addition illustrated by the following Figures and the Examples without in any way being limited by them.

### FIGURES

FIG. 1 illustrates a comparative study of the in vitro dissolution profile of the 67 mg fenofibrate tablets according to the invention and 67 mg fenofibrate tablets sold under the trade name Lipanthyl®.

FIG. 2 illustrates a comparative study of the in vitro dissolution profile of the 200 mg fenofibrate tablets according to the invention and 200 mg fenofibrate tablets sold under the trade name Lipanthyl®.

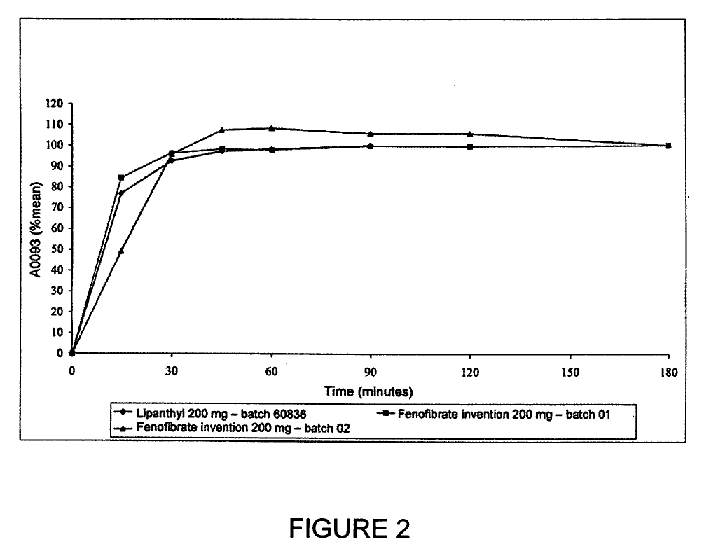

FIG. 3 illustrates a comparative study of the in vivo pharmacokinetic profile between 200 mg fenofibrate tablets according to the invention and 200 mg fenofibrate capsules sold under the trade name Lipanthyl®.

### EXAMPLES

### Material Used in the Examples

1—Description of the Material and the Manufacturing Procedure

Material

The whole material is available in accordance with the procedures in force at the site of manufacture, in conformity with Good Manufacturing Practices:

BÖHLE mixer (or equivalent)

ALPINE 200 AS micronizer (or equivalent)

ALEXANDER WERK granulator (or equivalent)

FREWITT MG 333 gauge (or equivalent)

KILLIAN TX26 rotatory compression machine (or equivalent)

18×8R8 format dies with comfort bar

WALTHER TROWAL film-coating turbine (or equivalent)

KLÖCKNER HÄNSEL packaging machine (or equivalent)

Description of the Manufacturing Procedure

All of the steps involved in the course of manufacture are conducted in conformity with Good Manufacturing Practices.

Step 0: Weighings

Carry out the weighings

Step 1: Mixture A

Introduce accurately weighed fenofibrate and sodium lauryl sulfate, if necessary sieved beforehand through a stainless steel sieve of mesh size 1.0 mm, into the interior of the mixer. Mix at about 6 rpm for about 30 minutes.

Step 2: Micronization

Micronize the mixture A for 40 minutes. The micronization parameters are:

| | |
|---|---|
| Air pressure: | 6 bars |
| Air flow: | 180 m³/h |
| Speed: | 25 kg/h |

Step 3: Mixture B

Introduce the co-micronizate, lactose monohydrate, anhydrous colloidal silica, type 102 microcrystalline cellulose and sodium croscarmellose, if necessary sieved beforehand through a stainless steel sieve with mesh size 1.0 mm into the interior of the mixer. Mix for about 20 minutes at 6 rpm. Add the first fraction of magnesium stearate. Mix for about 5 minutes at 6 rpm.

Step 4: Dry Granulation

Compress the mixture B by using the following parameters:
Force of compression 55 KN
Thickness of the compresses 1.25 mm
   Step 5: Calibration

Calibrate the bead obtained in the preceding step with a grid of mesh size 1.25 mm with the aid of the gauge.

Introduce the calibrated bead into the final mixer for homogenization. Mix for about 5 minutes at 6 rpm.

Step 6: Final Mixture

After having been sieved through a grid of diameter 1.0 mm, the remainder of the accurately weighed magnesium stearate is introduced into the mixer. Mix at about 6 rpm for 3 minutes.

Step 7: Compression

Equip the compression machine with adequate dies. Adjust the machine so as to obtain tablets in conformity with the following specifications:

TABLE 1

| | Specifications |
|---|---|
| Die format | 18 × 8R8 |
| Mean mass | 679.12 mg +/− 3% |
| Mass uniformity | Complies with |
| | Ph. Eur. (2.9.5) |
| Hardness on 10 uncoated | 60 to 100 N |
| tablets | |
| Friability on 20 uncoated | ≦1.00% |
| tablets | |
| Disintegration on 6 uncoated | ≦15 min |
| tablets | |

Steps 8 and 9: Film Coating

Introduce the necessary quantity of purified water for the preparation of a film-coating suspension dosed at 15% (m/m) into a recipient of adequate volume. Add accurately weighed white OPADRY® OYL 28900 to the purified water with shaking. Allow the suspension to stand for at least one hour with minimal shaking.

The quantity of film-coating solution can be adjusted as a function of the equipment used for the film-coating.

Introduce the uncoated tablets into the turbine and begin the film coating:

Temperature produced: 38° C.-40° C.

Air flow: 28 g/min

Spraying pressure: 2.5 bar

Turbine speed: 25 rpm

so as to obtain tablets in conformity with the following specifications:

TABLE 2

| | Specifications |
|---|---|
| Mean mass | 692.98 mg +/− 3% |
| Mass uniformity | Complies |
| | Ph. Eur. |
| | (2.9.5) |
| Hardness on 10 uncoated tablets | 70 to 110 N |
| Disintegration on 6 uncoated | ≦15 min |
| tablets | |

Step 10: Packaging

Package the tablets in a PVC/PVDC/Alu blister pack.

2—Control of the Critical and Intermediate Steps

Step 1: Mixture A

Verify the weighings and the control numbers of the starting materials

Verify the speed of rotation: about 6 rpm.

Verify the time of mixing: about 30 minutes

Verify the appearance of the mixture.

Step 2: Micronization

Verify the micronization parameters

Verify the size distribution of the co-micronizate:
75% of the particles have a size 1.75 μm±0.17 μm
25% of the particles have a size 4.7 μm±0.47 μm
90% of the particles have a size included between 0.824 μm±0.8 μm and 6.5 μm ±0.65 μm
d (0.5)=3.143 μm±0.31 μm
   Step 3: Mixture B

Verify the weighings and the control numbers of the starting materials

Verify the speed of rotation: about 6 rpm.

Verify the time of mixing: about 20 minutes

Verify the appearance of the mixture.

After the addition of magnesium stearate:

Verify the speed of rotation: about 6 rpm.

Verify the time of mixing: about 5 minutes

Verify the appearance of the mixture.

Step 4: Dry Granulation

Verify the compression parameters

Verify the appearance of the bead

Step 5: Calibration

Verify the conformity of the calibration grids (1.25 mm)

Verify the speed of rotation for homogenization: about 6 rpm.

Verify the time of homogenization: about 5 minutes

Step 6: Final Mixture

Verify the time and speed: about 3 minutes at 6 rpm.

Verify the appearance

Check the residual humidity [3 g-70° C.-15 min]: <10.0%

Step 7: Compression

Verify the die format: 18×8R8 with comfort bar

Verify the compression parameters periodically

Record the machine settings

Steps 8 and 9: Film Coating

Verify the speed, the time of preparation and the appearance (smooth and few air bubbles) of the film-coating solution

Verify and record the parameters of the film coating equipment

Verify the conformity of the film-coated tablets

Step 10: Packaging

Verify the conformity of the packaging materials

Verify the tightness of the seal

Verify the presence of regulatory information (batch number, expiry date).

### Example 1

### Preparation of Tablets Dosed at 67 mg of Fenofibrate

1.1 Qualitative and Quantitative Composition of the Tablets

The qualitative and quantitative composition of the tablets dosed at 67 mg of fenofibrate is presented in Table 3, at the end of the present description.

1.2 Manufacturing Procedure and Analysis

Description of the Manufacturing Procedure

All of the steps carried out in the course of manufacture are conducted in conformity with Good Manufacturing Practices

Verify the cleanliness of the equipment and of the work area

Step 0: Weighings

Carry out the weighings

Step 1: Mixture A

Introduce accurately weighed fenofibrate and sodium lauryl sulfate, if necessary sieved beforehand through a stainless steel sieve of mesh size 1.0 mm, into the interior of the mixer. Mix at about 6 rpm for about 30 minutes.

Step 2: Micronization

Micronize the mixture A for 40 minutes. The micronization parameters are:

| | |
|---|---|
| Air pressure: | 6 bars |
| Air flow: | 180 m³/h |
| Speed: | 25 kg/h |

Step 3: Mixture B

Introduce the co-micronizate, lactose monohydrate, anhydrous colloidal silica, type 102 microcrystalline cellulose and sodium croscarmellose, if necessary sieved beforehand through a stainless steel sieve with mesh size 1.0 mm, into the interior of the mixer. Mix for about 20 minutes at 6 rpm.

Add the first fraction of magnesium stearate. Mix for about 5 minutes at 6 rpm.

Step 4: Dry Granulation

Compress the mixture B by using the following parameters:

| | |
|---|---|
| Force of compression | 55 KN |
| Thickness of the compresses | 1.25 mm |

Step 5: Calibration

Calibrate the bead obtained in the preceding step with a grid of mesh size 1.25 mm with the aid of the gauge.

Introduce the calibrated bead into the final mixer for homogenization. Mix for about 5 minutes at 6 rpm.

Step 6: Final Mixture

After having been sieved through a grid of diameter 1.0 mm, the remainder of the accurately weighed magnesium stearate is introduced into the mixer. Mix at about 6 rpm for 3 minutes.

Step 7: Compression

Equip the compression machine with adequate dies. Adjust the machine so as to obtain tablets in conformity with the specifications presented in Table 4 below.

TABLE 4

| | Specifications |
|---|---|
| Die format | 10R10 |
| Mean mass | 284.37 mg ± 5% |
| Mass uniformity | Complies |
| | Ph. Eur. (2.9.5) |
| Hardness on 10 uncoated tablets | 60 N ± 20 N |
| Friability on 20 uncoated tablets | <1.0% |
| Disintegration on 6 uncoated tablets | <3 min |

Step 10: Packaging

Package the tablets in PVC/PVDC/Alu blister packs

Results for Fenofibrate Micronized with SLS

Coulter Size Distribution

Size Specifications of Co-Micronization:

100%<20 μm

50%<6 μm

20%<4 μm

Size distribution of the Bead by Superposed Sieves

The size distribution of the composition is shown in Table 5 below I

TABLE 5

| Mesh size | Rejects (%) | | | Cumulative rejects (%) | | |
|---|---|---|---|---|---|---|
| (μm) | 22404 | 22449 | 22450 | 22404 | 22449 | 22450 |
| 710 | 11.79 | 22.74 | 17.90 | 11.79 | 22.74 | 17.90 |
| 500 | 3.64 | 6.59 | 5.26 | 15.43 | 29.33 | 23.15 |
| 355 | 3.49 | 4.40 | 3.64 | 18.92 | 33.73 | 26.79 |
| 250 | 4.80 | 4.91 | 5.97 | 23.72 | 38.64 | 32.76 |
| 180 | 23.21 | 9.48 | 12.44 | 46.93 | 48.12 | 45.20 |
| 125 | 17.08 | 18.80 | 22.85 | 64.01 | 66.92 | 68.05 |
| 90 | 19.22 | 18.94 | 16.99 | 83.23 | 85.86 | 85.04 |
| Bottom | 16.77 | 14.14 | 14.96 | 100.00 | 100.00 | 100.00 |

Pharmacotechnical Characteristics—Final Mixture Before Compression

The pharmacotechnical characteristics of the composition are presented in Table 6 below.

TABLE 6

| TESTS | | Batch 01 | Batch 02 |
|---|---|---|---|
| Fluidity (s) (100 g) | | Infinite | Infinite |
| Apparent volume (ml) (100 g) | V0 | 242 | 234 |
| | V10 | 234 | 230 |
| | V500 | 178 | 175 |
| | V1250 | 178 | 169 |
| | V2500 | — | 169 |
| Compressibility (ml) | V10 − V500 | 56 | 55 |
| App. density (g/ml) | m/V0 | 0.41 | 0.43 |
| | m/V1250 | 0.56 | — |
| | m/V2500 | — | 0.59 |
| Residual moisture (%) | | 2.89 | 2.43 |
| (75% - 10 min) | | | |

Size Distribution through Superposed Sieves

The size distribution of the composition is given in Table 7 below.

TABLE 7

| | |
|---|---|
| Mesh size | Cumulative rejects in % |

| (μm) | Batch 01 | Batch 02 |
|---|---|---|
| 710 | 1.99 | 1.29 |
| 500 | 4.77 | 3.87 |
| 355 | 8.45 | 7.63 |
| 250 | 12.13 | 16.65 |
| 180 | 17.00 | 23.42 |
| 125 | 54.08 | 61.12 |
| 90 | 76.24 | 84.96 |
| Fond | 100.00 | 100.00 |

Pharmacotechnical Characteristics—Final Mixture Before Compression

The pharmacotechnical characteristics of the final mixture before compression are presented in Table 8 below.

TABLE 8

| TESTS | | Batch 01 | Batch 02 |
|---|---|---|---|
| Fluidity(s)* (100 g) | | 5.52 | 4.84 |
| Apparent volume (ml) (100 g) | V0 | 160 | 150 |
| | V10 | 152 | 140 |
| | V500 | 134 | 127 |
| | V1250 | 130 | 124 |
| | V2500 | 129 | 123 |
| Compressibility (ml) | V10 − V500 | 18 | 13 |
| App. density (g/ml) | m/V0 | 0.62 | 0.66 |
| | m/V1250 | 0.77 | 0.80 |
| | m/V2500 | 0.78 | 0.81 |
| Residual moisture (%) | | 2.80 | 2.24 |
| (75% - 10 min) | | | |

*The flow time measured in a glass funnel (cf. Ph. Eur. in force) brings to light electrostatic phenomena in the powder which disappear at the industrial level (STAINLESS STEEL feed). This phenomenon does not reflect an operational reality.

The size characteristics of the final mixture before compression are presented in Table 9 below

TABLE 9

| | |
|---|---|
| Mesh size | Cumulative rejects in % |

| (μm) | Batch 01 | Batch 02 |
|---|---|---|
| 710 | 22.61 | 32.77 |
| 500 | 32.07 | 42.93 |
| 355 | 37.75 | 48.80 |
| 250 | 43.53 | 53.78 |
| 180 | 48.90 | 58.07 |
| 125 | 59.86 | 66.63 |
| 90 | 68.82 | 74.10 |
| Bottom | 100.00 | 100.00 |

Results of the Pharmacotechnical Tests

The results of the pharmacotechnical tests are presented in Table 10, at the end of the present description.

Comparative in vitro Dissolution

The release profiles of fenofibrate in vitro have been compared between the tablets dosed at 67 mg of fenofibrate, batches No. 01 and No. 02, and prepared in conformity with the procedure according to the invention, and the tablets at the same dosage marketed under the trade name Lipanthyl®

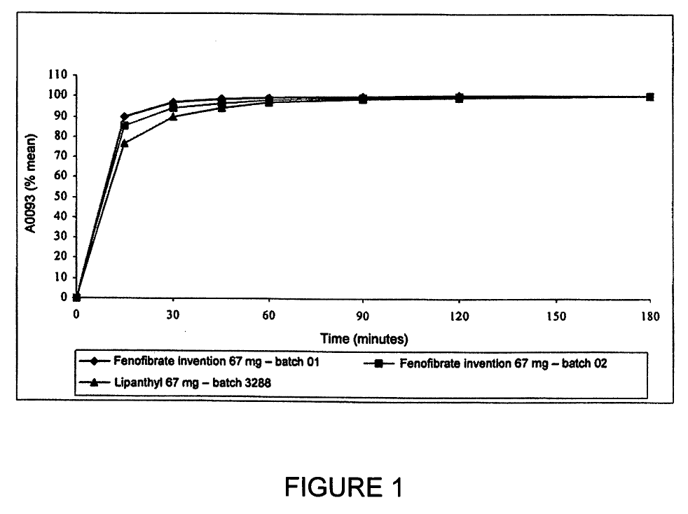

The results are presented in FIG. 1.

The tablets of the batches No. 01 and No. 02, prepared in conformity with the procedure of the invention, possess an in vitro dissolution profile of fenofibrate very similar, if not identical, to that of the Lipanthyl® 67 mg tablets.

### Example 2

### Preparation of Tablets Dosed at 200 mg of Fenofibrate

2.1 Qualitative and Quantitative Composition of the Tablets

Table 11: Qualitative and Quantitative Composition of the Tablets Dosed at 200 mg

The qualitative and quantitative composition of the tablets is presented in Table 11 below.

TABLE 11

| NAME | FORMULA | | | |
|---|---|---|---|---|
| OF THE CON- | Percent- | | | REFERENCE TO |
| STITUENTS | age | Unit | FUNCTION | STANDARDS |
| Active | | | | |
| ingredient | | | | |
| Fenofibrate | 23.56 | 200.00 | Active | Ph. Eur. 3rd |
| (micronized) | | | | edition Monograph |
| | | | | 1322 in force |
| Other | | | | |
| constituents | | | | |
| Sodium lauryl | 0.84 | 7.14 | Wetting | Ph. Eur. 3^{rd} edition |
| sulfate | | | agent | Monograph 0098 in |
| | | | | force |
| Lactose | 39.60 | 336.16 | Diluent | Ph. Eur. 3^{rd} edition |
| monohydrate | | | | Monograph 0187 in |
| | | | | force |
| Anhydrous | 0.50 | 4.24 | Glidant | Ph. Eur. 3^{rd} edition |
| colloidal silica | | | | Monograph 0434 in |
| | | | | force |
| Sodium | 5.00 | 42.44 | Disintegrant | Ph. Eur. 3^{rd} edition |
| croscarmellose | | | | Monograph 0985 in |
| | | | | force |
| Microcrystalline | 30.00 | 254.67 | Diluent/ | Ph. Eur. 3^{rd} edition |
| cellulose | | | Binder | Monograph 0316 |
| | | | | in force |
| Megnesium | 0.50 | 4.24 | Lubricant | Ph. Eur. 3^{rd} edition |
| stearate | | | | Monograph 0229 in |
| | | | | force |

| | |
|---|---|
| Unit mass (mg/tablet) | 848.90 |

2.2. Manufacturing Procedure and Analysis
Description of the Manufacturing Procedure

All of the steps carried out in the course of manufacture are conducted in conformity with Good Manufacturing Practices

Verify the cleanliness of the equipment and of the work area

Step 0: Weighings

Carry out the weighings

Step 1: Mixture A

Introduce accurately weighed fenofibrate and sodium lauryl sulfate, if necessary sieved beforehand through a stainless steel sieve of mesh size 1.0 mm, into the interior of the mixer. Mix at about 6 rpm for about 30 minutes.

Step 2: Micronization

Micronize the mixture A for 40, minutes. The micronization parameters are:

| | |
|---|---|
| Air pressure: | 6 bars |
| Air flow: | 180 m³/h |
| Speed: | 25 kg/h |

Step 3: Mixture B

Introduce the co-micronizate, lactose monohydrate, anhydrous colloidal silica, type 102 microcrystalline cellulose and sodium croscarmellose, if necessary sieved beforehand through a stainless steel sieve with mesh size 1.0 mm, into the interior of the mixer. Mix for about 20 minutes at 6 rpm.

Add the first fraction of magnesium stearate. Mix for about 5 minutes at 6 revs/min.

Step 4: Dry Granulation

Compress the mixture B by using the following parameters:

| | |
|---|---|
| Force of compression | 55 KN |
| Thickness of the compresses | 1.25 mm |

Step 5; Calibration

Calibrate the bead obtained in the preceding step with a grid of mesh size 1.25 mm with the aid of the gauge.

Introduce the calibrated bead into the final mixer for homogenization. Mix for about 5 minutes at 6 rpm.

Step 6: Final Mixture

After having been sieved through a grid of diameter 1.0 mm, the remainder of the accurately weighed magnesium stearate is introduced into the mixer. Mix at about 6 rpm for 3 minutes.

Step 7: Compression

Equip the compression machine with adequate dies. Adjust the machine so as to obtain tablets in conformity with the specifications presented in Table 20 below.

TABLE 12

| | Specifications |
|---|---|
| Die format | 14R15 |
| Mean mass | 848.90 mg ± 5% |
| Mass uniformity | Complies |
| | Ph. Eur. (2.9.5) |
| Hardness on 10 uncoated tablets | 70 N ± 30 N |
| Friability on 20 uncoated tablets | <0.5% |
| Disintegration on 6 uncoated tablets | <3 min |

Step 9: Packaging

Package the tablets in a PVC/PVDC/Alu blister pack.

Results for Fenofibrate

Pharmacotechnical Characteristics of the Bead

The pharmacotechnical characteristics of the bead are presented in Table 21 below

TABLE 13

| | | Batch | Batch | Batch |
|---|---|---|---|---|
| TESTS | | 22404 | 22449 | 22450 |
| Fluidity (100 g) | | Infinite | Infinite | Infinite |
| Volume apparent (100 g): | V0 | 164 ml | 149 ml | 156 ml |
| | V10 | 158 ml | 148 ml | 152 ml |
| | V500 | 138 ml | 129 ml | 134 ml |
| | V1250 | 137 ml | 128 ml | 132 ml |
| Compressibility | V10 − V500 | 20 ml | 19 ml | 18 ml |
| Apparent density | m/V0 | 0.614 | 0.669 | 0.640 |
| (g/ml) | m/V1250 | 0.736 | 0.779 | 0.757 |
| Residual moisture (%) | | 2.39 | 2.25 | 1.87 |
| (3 g - 15 min - 70° C.) | | | | |

Size Distribution of the Bead through Superposed Sieves

The size distribution of the composition is presented in Table 14 below

TABLE 14

| Mesh size | Rejects (%) | | | Cumulative rejects (%) | | |
|---|---|---|---|---|---|---|
| (μm) | 22404 | 22449 | 22450 | 22404 | 22449 | 22450 |
| 710 | 11.79 | 22.74 | 17.90 | 11.79 | 22.74 | 17.90 |
| 500 | 3.64 | 6.59 | 5.26 | 15.43 | 29.33 | 23.15 |
| 355 | 3.49 | 4.40 | 3.64 | 18.92 | 33.73 | 26.79 |
| 250 | 4.80 | 4.91 | 5.97 | 23.72 | 38.64 | 32.76 |
| 180 | 23.21 | 9.48 | 12.44 | 46.93 | 48.12 | 45.20 |
| 125 | 17.08 | 18.80 | 22.85 | 64.01 | 66.92 | 68.05 |
| 90 | 19.22 | 18.94 | 16.99 | 83.23 | 85.86 | 85.04 |
| Bottom | 16.77 | 14.14 | 14.96 | 100.00 | 100.00 | 100.00 |

Pharmacotechnical Characteristics—Final Mixture Before Compression

The pharmacotechnical characteristics of the final mixture are presented in Table 15 below.

TABLE 15

| TESTS | | Batch 01 | Batch 02 |
|---|---|---|---|
| Fluidity (s) (100 g) | | Infinite | Infinite |
| Apparent volume (ml) (100 g) | V0 | 242 | 234 |
| | V10 | 234 | 230 |
| | V500 | 178 | 175 |
| | V1250 | 178 | 169 |
| | V2500 | — | 169 |
| Compressibility (ml) | V10 − V500 | 56 | 55 |
| Apparent density(g/ml) | m/V0 | 0.41 | 0.43 |
| | m/V1250 | 0.56 | — |
| | m/V2500 | — | 0.59 |
| Residual humidity (%) | | 2.89 | 2.43 |
| (75% - 10 min) | | | |

Size Distribution through Superposed Sieves

The size distribution of the composition is presented in Table 16 below.

TABLE 16

| | |
|---|---|
| Mesh size | Cumulative rejects in % |

| (μm) | Batch 01 | Batch 02 |
|---|---|---|
| 710 | 1.99 | 1.29 |
| 500 | 4.77 | 3.87 |
| 355 | 8.45 | 7.63 |
| 250 | 12.13 | 16.65 |
| 180 | 17.00 | 23.42 |
| 125 | 54.08 | 61.12 |
| 90 | 76.24 | 84.96 |
| Bottom | 100.00 | 100.00 |

Pharmacotechnical Characteristics—Final Mixture Before Compression

The pharmacotechnical characteristics of the final mixture are presented in Table 17 below.

TABLE 17

| | | Batch | Batch |
|---|---|---|---|
| Tests | | 01 | 02 |
| Fluidity (s)* (100 g) | | 5.52 | 4.84 |
| Apparent volume (ml) (100 g) | V0 | 160 | 150 |
| | V10 | 152 | 140 |
| | V500 | 134 | 127 |
| | V1250 | 130 | 124 |
| | V2500 | 129 | 123 |
| Compressibility (ml) | V10 − V500 | 18 | 13 |
| Apparent density (g/ml) | m/V0 | 0.62 | 0.66 |
| | m/V1250 | 0.77 | 0.80 |
| | m/V2500 | 0.78 | 0.81 |
| Residual moisture (%) | | 2.80 | 2.24 |
| (75% - 10 min) | | | |

*The flow time measured in a glass funnel (cf. Ph. Eur. in force) brings to light electrostatic phenomena in the powder which disappear at the industrial level (STAINLESS STEEL feed). This phenomenon does not reflect an operational reality.

Size Distribution through Superposed Sieves

The size distribution is presented in Table 18 below

TABLE 18

| | |
|---|---|
| Mesh size | Cumulative rejects in % |

| (μm) | Batch 01 | Batch 02 |
|---|---|---|
| 710 | 22.61 | 32.77 |
| 500 | 32.07 | 42.93 |
| 355 | 37.75 | 48.80 |
| 250 | 43.53 | 53.78 |
| 180 | 48.90 | 58.07 |
| 125 | 59.86 | 66.63 |
| 90 | 68.82 | 74.10 |
| Bottom | 100.00 | 100.00 |

The results of the controls of the fenofibrate tablets are presented in Table 27, at the end of the present description.

Comparative in vitro Dissolution

The release profiles of fenofibrate in vitro have been compared between the tablets dosed at 200 mg of fenofibrate, batches No. 01 and No. 02, and prepared in conformity with the procedure according to the invention, and the tablets at the same dosage marketed under the trade name Lipanthyl®

The results are presented in FIG. 6.

The tablets of the batches No. 01 and No. 02, prepared in conformity with the procedure of the invention, possess an in vitro dissolution profile of fenofibrate very similar, if not identical, to that of the Lipanthyl® 200 mg tablets.

### Example 3

### Comparative Analysis of in vivo Pharmacokinetics Between Fenofibrate Tablets Dosed at 200 mg According to the Invention and Fenofibrate Capsules Marketed Under the Trade Name Lipanthyl® 200M at the Same Dosages

A group of 23 individuals, 17 men and 6 women between 18 and 40 years of age, were selected for the study and were divided into two subgroups: a first subgroup treated with fenofibrate tablets dosed at 200 mg according to the invention and a second subgroup treated with fenofibrate tablets dosed at 200 mg and marketed under the trade name Lipanthyl®.

After breakfast each individual took a 200 mg tablet according to the invention or a 200 mg Lipanthyl® tablet by the oral route with 240 ml of water.

Then, 10 ml samples of venous blood were collected from each individual in heparinized glass tubes, before the oral ingestion of the fenofibrate tablet and at the times 1, 2, 3, 4, 4.5, 5, 5.5, 6, 7, 8, 10, 12, 16, 24, 48, 72 and 96 hours after ingestion of the fenofibrate tablet.

The concentration of fenofibric acid, expressed in ng/ml, was measured in each blood sample taken.

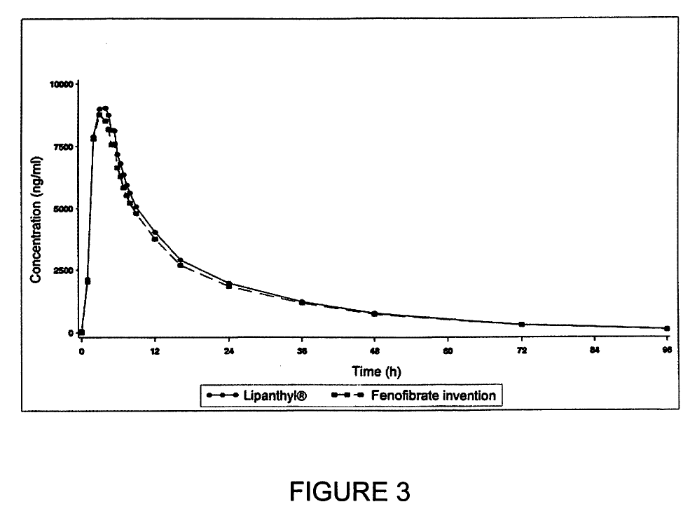

The mean concentration of fenofibric acid for all of the individuals of each of the two groups was calculated. The results are presented in FIG. 3.

The results of FIG. 3 show that the in vivo pharmacokinetic profile of fenofibrate for the individuals being treated with 200 mg fenofibrate tablets according to the invention is identical with the pharmacokinetic profile of fenofibrate for the individuals treated with the Lipanthyl® tablets dosed at 200 mg.

### Example 4

### Comparative Analysis of in vivo Pharmacokinetics Between Fenofibrate Tablets Dosed at 200 mg According to the Invention and Fenofibrate Capsules Marketed Under the Trade Name Lipanthyl® at the Same Dosage

A group of 26 individuals, 12 men and 14 women, respectively, between 18 and 55 years of age, were selected for a study comprising two treatment steps of 96 hours each, i.e. a treatment step with fenofibrate tablets dosed at 200 mg according to the invention and a treatment step with fenofibrate capsules dosed at 200 mg and marketed under specialty products of the trade name Lipanthyl® 200 mg. The two treatment steps were separated by a 14-day period of time taking into account the fenofibric acid half-life. Treating sequence for each patient was randomized.

Each patient has taken a fat-rich and energetic breakfast and five minutes after end of it, took a 200 mg fenofibrate tablet according to the invention and a 200 mg Lipanthyl® capsule by the oral route with 180 ml of water.

Then, 10 ml samples of venous blood were collected from each individual in heparinized glass tubes, respectively before the oral ingestion of the fenofibrate tablet or of the fenofibrate capsule and at the times 1, 2, 3, 4, 4.5, 5, 5.5, 6, 6.5, 7, 7.5, 8, 9, 10, 12, 16 and 24 hours after ingestion of the fenofibrate tablet or capsule.

The concentration of fenofibric acid, as expressed in ng/ml, was measured in each blood sample taken.

The arithmetic mean concentration of fenofibric acid varying with time was also calculated for all of the individuals after the treatment step with the 200 mg fenofibrate tablets according to the invention and after the treatment step with the Lipanthyl® capsules dosed at 200 mg.

The variation curve of plasma fenofibric acid arithmetic mean concentration versus time was also plotted.

The maximum plasma concentration (Cₘₐₓ), the time until the maximum plasma concentration has been reached (Tₘₐₓ) and the area under the plasma concentration curve (AUC) have been measured for each treatment.

The values for these variables, means and ranges thereof were calculated for each treatment step and presented in following Table 20 below.

TABLE 20

| | | 200 mg | 200 mg capsule | | |
|---|---|---|---|---|---|
| | | tablet of the | Arrow ® | 200 mg capsule | 160 mg tablet |
| Parameter | | invention | Génériques | Lipanthyl ® | Lipanthyl ® |
| Cₘₐₓ | mean | 10670 | 10384 | 13084 | 10592 |
| | standard | 1988 | 2148 | 2087 | — |
| | deviation | | | | |
| Tₘₐₓ | mean | 5.25 | 5.50 | 5.50 | — |
| | standard | — | — | — | — |
| | deviation | | | | |
| AUC₀₋ₜ | mean | 219718 | 205781 | 237068 | — |
| | standard | 69070 | 65951 | 65131 | — |
| | deviation | | | | |
| AUC_{0-∞} | mean | 230144 | 213902 | 247157 | 167702 |
| | standard | 79070 | 73066 | 77619 | — |
| | deviation | | | | |

AUC_{0-t:} area under the curve for plasma concentration as measured between 0 and 24 hours.
AUC_{0-∞:} area under the curve for plasma concentration as calculated by extrapolation to infinite.

Table 20 variables were compared to table 21 below:

TABLE 21

| | Tablet | | |
|---|---|---|---|
| | of the invention - | 200 mg capsule | |
| Parameter | 90% CI* - | Lipanthyl ® - 90% CI - | Result |
| Cₘₐₓ | 74.6-88.2 | 72.5-85.6 | NS* |
| Tₘₐₓ | NS | NS | NS |
| AUC₀₋ₜ | 86.3-97.1 | 80.7-90.8 | NS |
| AUC_{0-∞} | 86.7-97.7 | 80.6-90.8 | NS |

90% CI*: 90% confidence interval.
NS*: not statistically different.

The combined results of table 21 hereabove show that the in vivo pharmacokinetic profile of fenofibrate for individuals treated according to the treatment step with the 200 mg fenofibrate tablets according to the invention is the same as the pharmacokinetic profile of fenofibrate for individuals treated according to the treatment step with the Lipanthyl® capsules dosed at 200 mg.

TABLE 3
qualitative and quantitative composition of the 67 mg tablets

| NAME | FORMULA | | | |
|---|---|---|---|---|
| OF THE CON- | Percent- | | | REFERENCE TO |
| STITUENTS | age | Unit | FUNCTION | STANDARDS |
| Active | | | | |
| ingredient | | | | |
| Fenofibrate | 23.56 | 67.00 | Active | Ph.Eur. |
| (micronized) | | | | Monograph 1322 |
| Other | | | | |
| constituents | | | | |
| Sodium lauryl | 0.84 | 2.39 | Wetting | Ph.Eur |
| sulfate | | | agent | Monograph 0098 |
| Lactose | 39.60 | 112.61 | Diluent | Ph.Eur. |
| monohydrate | | | | Monograph 0187 |
| Anhydrous | 0.50 | 1.42 | Glidant | Ph.Eur. |
| colloidal silica | | | | Monograph 0434 |
| Sodium | 5.00 | 14.22 | Disintegrant | Ph.Eur. |
| croscarmellose | | | | Monograph 0985 |
| Microcrystalline | 30.00 | 85.31 | Diluent/ | Ph.Eur. |
| cellulose | | | Binder | Monograph 0316 |
| Magnesium | 0.50 | 1.42 | Lubricant | Ph.Eur. |
| stearate | | | | Monograph 0229 |

| | |
|---|---|
| Unit mass (mg/tablet) | 284.38 |

TABLE 10
Results of the pharmacotechnical tests for the fenofibrate tablets dosed at 67 mg

| | | Results | | | | | |
|---|---|---|---|---|---|---|---|
| | | batch 01 | | | batch 02 | | |
| Controls | Standards | Start | Middle | End | Start | Middle | End |
| Characters | | | | | | | |
| Appearance | round | complies | complies | complies | complies | complies | complies |
| Colour | white | complies | complies | complies | complies | complies | complies |
| Odour | odourless | complies | complies | complies | complies | complies | complies |
| Tests | tablet | | | | | | |
| Mass uniformity | | | | | | | |
| tablet | | complies | complies | complies | complies | complies | complies |
| CV (%) | complies | 2.60 | 2.43 | 2.28 | 2.43 | 2.13 | 1.90 |
| Mean mass (mg) | | | | | | | |
| tablet | | 285.05 | 286.23 | 280.30 | 286.36 | 286.40 | 284.35 |
| Disintegration (min) | 270-298.6 | 1 min 37 | 1 min 06 | 1 min 05 | 41 s | 47 s | 45 s |
| Hardness (N) | <5 min | 77.60 | 55.20 | 46.80 | 51.40 | 45.60 | 49.50 |
| Uniformity of content | 60 ± 20 N | complies | complies | complies | complies | complies | complies |
| mean | | 61.78 | 65.40 | 66.47 | 66.99 | 65.36 | 69.15 |
| CV % | | 4.83 | 2.35 | 2.82 | 6.22 | 2.66 | 3.31 |
| Dissolution in vitro at 30 min | CV < 10% | complies | complies | complies | complies | complies | complies |
| Release in SLS 2% | | | | | | | |
| Identification and | >75% | | | | | | |
| dosage | | | | | | | |
| Identification of | control tr = test tr | complies | complies | complies | complies | complies | complies |
| Fenofibrate | | | | | | | |
| Dosage of Fenofibrate | 63.65 to | | 64.59 | | | 66.90 | |
| (mg/tab) | 70.35 | | | | | | |
| Supplementary tests | | | | | | | |
| Residual moisture (%) | <5 | 2.67 | 2.37 | 2.34 | 2.75 | 2.55 | 2.47 |
| Friability (%) | <0.5 | 0.16 | 0.16 | 0.095 | 0.34 | 0.26 | 0.43 |

TABLE 19
Results of the controls of the fenofibrate tablets according to the invention dosed at 200 mg

| | | Results | | | | | |
|---|---|---|---|---|---|---|---|
| | | Batch 01 | | | Batch 02 | | |
| Controls | Standards | Start | Middle | End | Start | Middle | End |
| Characters | | | | | | | |
| Appearance | _round tab. | complies | complies | compiies | complies | complies | complies |
| Colour | white | complies | complies | complies | complies | complies | complies |
| Odour | odourless | complies | complies | complies | complies | complies | complies |
| Tests | | | | | | | |
| Mass uniformity | | | | | | | |
| tablet | complies | complies | complies | complies | complies | complies | complies |
| CV (%) | | 1.17 | 0.73 | 1.25 | 0.61 | 0.73 | 0.76 |
| Mean mass (mg) | | | | | | | |
| tablet | 806-891 | 843.48 | 854.89 | 861.42 | 848.18 | 849.63 | 849.41 |
| Disintegration (min) | <3 min | 1 min 16 | 1 min 20 | 1 min 20 | 1 min 06 | 1 min 09 | 1 min 22 |
| Hardness (N) | 70 ± 30 N | 48.70 | 60.90 | 63.20 | 79.20 | 86.10 | 83.00 |
| Uniformity of content | | complies | complies | complies | complies | complies | complies |
| Mean | | 201.79 | 192.55 | 187.79 | 198.07 | 188.59 | 191.95 |
| CV % | CV < 10% | 5.09 | 1.80 | 6.79 | 5.81 | 6.00 | 6.78 |
| Dissolution in vitro at 30 min* | >75% | complies | complies | complies | complies | complies | complies |
| Release in SLS 2% | | | | | | | |
| Identification and dosage | | | | | | | |
| Identification of Fenofibrate | control tr = test tr | complies | complies | complies | complies | complies | complies |
| Dosage of Fenofibrate (mg/tab) | 190.0 to 210.0 | | 193.9 | | | 199.1 | |
| Supplementary tests | | | | | | | |
| Residual moisture (%) | <5 | 2.97 | 3.07 | 2.70 | 2.54 | 2.50 | 2.58 |
| Friability (%) | <0.5 | 0.12 | 0.23 | 0.11 | 0.34 | 0.28 | 10.24 |

1. A method for the manufacture of a pharmaceutical composition containing the active ingredient fenofibrate or one of its derivatives, optionally in the form of a combination of fenofibrate or its derivative with a second active ingredient, in the form of tablets, characterized in that it comprises the following steps:
(a) prepare a mixture of fenofibrate or one of its derivatives, or of the combination with a second active ingredient, and at least one solid surfactant in a ratio of (i) 91% to 99% by weight of fenofibrate or its derivative and (ii) 1% to 9% by weight of the said solid surfactant(s);
(b) micronize the mixture of fenofibrate or one of its derivatives, optionally in the form of a combination with a second active ingredient, and surfactants) obtained in step (a) in order to obtain a micronizate of fenofibrate or of the combination with the second active ingredient, and the surfactant(s);
(c) add at least one anti-static agent to the co-micronizate prepared in step (b);
(d) add to the mixture obtained in step (c) at least one diluent, at least one disintegrant and at least one lubricant in order to obtain a solid mixture corresponding to the internal phase of the tablet;
(e) compress the solid mixture obtained in step (d) through a dry granulation step, in order to obtain the final internal phase of the tablet;
(f) mix the internal phase of the tablet prepared in step (e) with an external phase comprising at least one lubricant, then carry out a compression of the composition in order to obtain the pharmaceutical composition containing fenofibrate or one of its derivatives in the form of tablets.

2. The method of claim 1, wherein in step (a), the mixture of fenofibrate or one of its derivatives, optionally in the form of a combination with a second active ingredient, and surfactant(s) comprises a ratio of (i) 95% to 98% by weight of fenofibrate or the combination and (ii) 2% to 5% by weight of surfactant(s).

3. The method according to anyone of claims 1 or 2, wherein in step (b), the co-micronizate consists of particles possessing a size included between 0.1 and 20 μm.

4. The method according to claim 1, wherein in step (c) the anti-static agent(s) is/are added in an amount of 0.1% to 5% by weight with respect to the total weight of the composition.

5. The method according to claim 1, wherein in step (d) the diluent(s) is/are added in an amount of 40% to 80% by weight, with respect to the total weight of the composition.

6. The method according to claim 1, wherein in step (d) the disintegrant(s) is/are added in an amount of 1% to 20% by weight, with respect to the total weight of the composition.

7. The method according to claim 1, wherein in step (d) the lubricant(s) is/are added in an amount of 0.1% to 2% by weight, with respect to the total weight of the composition.

8. The method according to claim 1, wherein the fenofibrate or its derivative is present in an amount of 20% to 50% by weight, with respect to the total weight of the composition.

9. The method of claim 8, wherein the fenofibrate or its derivative is combined with a second active ingredient.

10. The method of claim 9, wherein said second active ingredient is selected from metformin, cobalamine, folic acid, betaine, N-acetylcysteine, vitamin E and an inhibitor of HGMCoA.

11. The method according to claim 1, wherein the surfactant(s) is/are selected from the following surfactants: sodium lauryl sulfate, a polyoxyethylenated ester of polysorbitan, such as the monooleate, monolaurate, monopalmitate, monostearate esters, sodium dioctylsulfosuccinate (DOSS) and lecithin.

12. The method according to claim 1, wherein the anti-static agent(s) is/are selected from colloidal silica, magnesium silicate, talc, calcium silicate and tribasic calcium phosphate.

13. The method according to claim 1, wherein the diluent(s) is/are selected from calcium or sodium carbonate or bicarbonate, sucrose, mannitol, xylitol, sorbitol, lactose, maltitol, glucose, cellulose powder or microcrystalline cellulose, starch and its derivatives, dibasic calcium phosphate, tribasic calcium phosphate, calcium sulfate, the dextrates, the dextrins, dextrose excipients, fructose, kaolin, lactitol.

14. The method according to claim 1, wherein the disintegrant(s) is/are selected from sodium starch glycollate, sodium croscarmellose, cross-linked polyvinylpyrrolidone, sodium carboxymethylcellulose and calcium carboxymethylcellulose and lightly substituted hydroxypropylcellulose.

15. The method according to claim 1, wherein the lubricant(s) is/are selected from magnesium stearate, calcium stearate and talc.

16. The method according to claim 1, wherein said method further comprises an additional film-coating step (g) of the tablet obtained in step (f).

17. A tablet of fenofibrate or one of its derivatives, optionally in combination with a second active ingredient, wherein said tablet comprises:
(a) an internal phase consisting of:
(i) 20% to 50% by weight of fenofibrate or one of its derivatives and optionally a second active ingredient, in the form of a micronizate with at least one solid surfactant, said micronizate comprising (i) from 91% to 99% by weight of fenofibrate or one of its derivatives, optionally in combination with the second active ingredient, and (ii) from 1% to 9% by weight of the said solid surfactant(s);
(ii) from 0.2% to 2% by weight of at least one anti-static agent; (iii) from 40% to 80% by weight of at least one diluent;
(iv) from 1% to 20% by weight of at least one disintegrant; and (v) from 0.1% to 1% of at least one lubricant; with respect to the total weight of the said tablet; and
(b) an external phase comprising from 0.1% to 2% at least one lubricant with respect to the total weight of the said tablet.

18. The tablet of claim 17, wherein the external phase is covered with a protective varnish, preferably a protective varnish based on a hydrodispersible polymer.

19. The tablet of claim 17 in which the fenofibrate or its derivative is combined with a second active ingredient.

20. The tablet of claim 17, wherein the second active ingredient is selected from mefformin, cobalamine, folic acid, betaine, N-acetylcysteine, vitamin E and an inhibitor of HGMCoA.

21. The tablet according to anyone of claims 17-20, dosed at 200 mg, which pharmacokinetic profile is characterized by an area under the plasma concentration curve measured in vivo (AUC) of about 220000 ng.h/ml.

22. The tablet according to anyone of claims 17-20, dosed at 200 mg, which pharmacokinetic profile is characterized by a maximum plasma concentration value (Cₘₐₓ) of about 10600 ng/ml.

23. The tablet according to anyone of claims 17-20, dosed at 200 mg, which pharmacokinetic profile is characterized by a Tₘₐₓ ranging from 2.00 to 3.75 ng/ml.

24. The method according to claim 4, wherein in step (c) the anti-static agent(s) is/are added in an amount of 0.2% to 2% by weight with respect to the total weight of the composition.

25. The method according to claim 5, wherein in step (d) the diluent(s) is/are added in an amount between 50% and 75% by weight, with respect to the total weight of the composition.

26. The method according to claim 6, wherein in step (d) the disintegrant(s) is/are added in an amount of from 3 to 6% by weight, with respect to the total weight of the composition.

27. The method according to claim 7, wherein in step (d) the lubricant(s) is/are added in an amount of from 0.2% to 1% by weight, with respect to the total weight of the composition.
